(19) United States
(12) Patent Application Publication
(10) Pub. No.: US 2020/0315233 A1
(43) Pub. Date: Oct. 8, 2020

(54) METHODS AND COMPOSITIONS FOR TREATMENT OF HYPERCALCIURIA AND NEPHROLITHIASIS

(71) Applicant: Ana Pharmaceuticals, Inc., San Juan, PR (US)

(72) Inventor: Eduardo I. Canto, San Juan, PR (US)

(73) Assignee: Ana Pharmaceuticals, Inc., San Juan, PR (US)

(21) Appl. No.: 16/305,040

(22) PCT Filed: Jun. 1, 2017
(86) PCT No.: PCT/US2017/035514

§ 371 Date: Nov. 27, 2018

Related U.S. Application Data
(60) Provisional application No. 62/344,653, filed on Jun. 2, 2016.

(51) Int. Cl. A23L 33/15; A23L 33/00; A61K 31/122; A61K 31/194; A61K 33/08; A61K 31/675; A61K 33/00; A61P 13/04

(52) U.S. Cl. CPC A23L 33/15; A23L 33/40; A61K 31/122; A61K 31/194; A23V 2002/00; A61K 31/675; A61K 33/00; A61P 13/04; A61K 33/08

(57) ABSTRACT

Disclosed are methods, compositions and formulations for treating or preventing hypercalciuria thereby reducing the risk of nephrolithiasis and normalizing urine chemistry and composition. The method includes administering vitamin K2 in a medicinal or nutritional composition, preferably without calcium, optionally in combination with a therapeutic dose of one or more selected from the group consisting of: a citrate salt, magnesium oxide, a bicarbonate salt and vitamin B6.

### RELATED APPLICATIONS

This application is an International Patent Application that claims the benefit of priority under 35 U.S.C. § 119(e) of U.S. Provisional Application No. 62/344,653, filed on Jun. 2, 2016, which is incorporated herein by reference in its entirety.

### FIELD

The present invention relates to a use of vitamin K2 for treating or preventing hypercalciuria and related diseases and preventing kidney stone recurrence.

### BACKGROUND

Nephrolithiasis results from the precipitation of crystal aggregates in the upper urinary tract. Hypercalciuria or hypercalcinuria is the condition of elevated calcium in the urine. Chronic hypercalciuria may lead to nephrolithiasis, impairment of function, nephrocalcinosis, and renal insufficiency. Patients with hypercalciuria have kidneys that filter abnormally high levels of calcium.

About 33% of patients with nephrolithiasis have hypercalciuria. The current standard of care treatment for idiopathic hypercalciuria in patients with nephrolithiasis is restriction of dietary sodium and protein, and off-label use of prescription thiazide diuretics. Thiazide diuretics are fraught with side effects and, therefore, are rarely prescribed. Furthermore, when they are prescribed, patients rarely adhere to treatment

It would be desirable to have new medicinal or nutritional therapies for kidney stone prevention. It also would be desirable to have new medicinal or nutritional treatments for hypercalciuria.

### SUMMARY

The invention is based, at least in part, upon discovery of new medicinal and nutritional compositions and methods for treatment of hypercalciuria and nephrolithiasis.

In particular, administration of vitamin K2 to human subjects was identified to significantly reduce urinary calcium, including administration to human subjects identified as suffering from calcium nephrolithiasis and hypercalciuria. Many patients experienced a decrease of more than 40% in urine calcium levels within a month of therapy in accordance with the present invention. Additionally, the decrease in calcium levels provided by administration of vitamin K2 in accordance with the invention may not be significantly affected by other therapies that may be directed at reducing other risk factors for recurrent nephrolithiasis.

Thus, in one aspect, methods for treating hypercalciuria are provided that comprise administering an effective amount of vitamin K2 to a subject. Optionally, the subject is a mammal.

In a further aspect, methods are provided for treating hypercalciuria and/or nephrolithiasis in a mammalian subject, that comprise administering to the subject an effective amount of vitamin K (i.e., K1 and/or K2) in conjunction with one or more of a citrate salt, magnesium oxide, vitamin B6, or a bicarbonate salt. In certain embodiments, the subject may be predisposed to nephrolithiasis. Optionally, the mammalian subject is identified as having hypercalciuria.

In an additional aspect, methods are provided for treating nephrolithiasis comprising administering to a subject an effective amount of vitamin K (i.e., K1 and/or K2).

In certain embodiments of all the present treatment methods, an effective amount of MK4 is administered to the subject. In additional embodiments of all the treatment methods, an effective amount of MK7 is administered to the subject. In further embodiments, vitamin K1 is co-administered to the subject.

In certain methods, vitamin K2 is administered to the subject in conjunction with one or more of a citrate salt, magnesium oxide, vitamin B6, or a bicarbonate salt.

In certain methods, vitamin K2 is administered together with one or more potassium-containing agents to deliver a relatively high content of potassium to a subject, such as 100 mg or greater of potassium to the subject per dose. The potassium source may be a variety of potassium-containing compositions such as potassium citrate and others. Such methods and related compositions can be particularly effective for treating renal tubular acidosis including with associated stones.

In certain preferred methods, one or more vitamin K compounds are administered in the substantial absence of calcium, i.e. the therapeutic composition(s) administered to a subject will be at last substantially free of any calcium-containing agents, i.e. the amount of calcium as a component of a present vitamin K composition in a daily dose will be less than 50 mg, more typically less than 40, 30, 20, 10, 5 or even 1 mg in a daily dose. Certain preferred vitamin K compositions as disclosed herein will be effectively free of any calcium-containing agents. In the present methods, a subject for treatment is optionally identified and selected for treatment prior to administration of a therapeutic composition as disclosed herein. For instance, a human subject may be selected for treatment based on levels of urine calcium secretion, e.g. selecting a human subject that excretes greater than 200 mg of calcium per 24 hour period and administering to that selected subject an effective amount of a therapeutic agent (e.g., vitamin K2) as disclosed herein.

Additionally, in certain methods, the treated subject may be assessed during the course of treatment or periodically post-treatment, for example, monitoring urine calcium secretion of the subject in conjunction with administering vitamin K (i.e., K1 and/or K2), including post-administration.

Optionally, vitamin K is administered in an oral dosage form. In certain embodiments, the administered vitamin K has been isolated, i.e. it is separate and isolated from any naturally occurring source. The administered vitamin K2 also may be synthetically derived. Oral dosage forms such as tablets and capsules are often utilized.

In one embodiment, the subject is identified as suffering from one or more of the following: hypercalciuria, hyperuricosuria, hyperoxaluria, hypocitraturia, or hypomagnesiuria.

In certain embodiments, the subject is not receiving anticoagulant therapy and is not suffering from osteoporosis or neoplasia. In related embodiments, the subject is not receiving anticoagulant therapy and has not been identified as suffering from or susceptible to osteoporosis or neoplasia.

Therapeutic compositions are also provided. In one aspect, a therapeutic composition is provided that includes an effective amount of vitamin K (i.e., K1 and/or K2) in combination with potassium citrate, magnesium citrate, and vitamin B6. In certain embodiments the composition contains an effective amount of MK4 and/or MK7. The composition also may optionally comprise vitamin K1.

In a further aspect, high potassium content compositions that comprise vitamin K2 are provided. Such compositions may be useful for treatment of among others renal tubular acidosis including with associated stones. Preferably, the compositions are in a form where 90, 95, 99 or 100 mg or greater of potassium may be conveniently administered to a human subject per unit dose (such as an oral dosage form e.g. tablet or capsule) and/or each day. For instance, for an oral dosage form such as a capsule or tablet, the dosage form may contain at least 10, 20, 30, 40, 50, 60, 70, 80, 90, 95, 99 or 100 mg of potassium to provide for convenient dosing of the human subject. A variety of potassium sources may be utilized, including for example potassium citrate. In a particular aspects, compositions including in oral dosage forms such as tablets or capsules are provided that comprise in dosage amounts 1) an effective amount of vitamin K (vitamin K1 and/or vitamin K2) and 1) potassium citrate in an amount of 540 mg or greater. In an additional aspect, compositions including in oral dosage forms such as one or more tablets or capsules are provided that comprise in dosage amounts 1) an effective amount of vitamin K2; 2) vitamin B6 in an amount greater than 7.5 mg; 3) magnesium citrate in an amount greater than 180 mg; and 4) potassium (such as in the form of potassium citrate) in an amount not exceeding 100 or 99 mg. Amounts of potassium without further limitation by as referred to herein designate the amount of K or K⁺ in a therapeutic or nutritional composition rather than the amount of a potassium complexed compound such as potassium citrate. On the other hand, if an amount of the complexed form is specified (e.g. potassium citrate), then the amount refers to the complexed form (e.g potassium citrate) rather than just the amount of K or K⁺ in a therapeutic or nutritional composition.

In a yet further aspect, compositions including in oral dosage forms such as tablets or capsules are provided that comprise 1) an effective amount of vitamin K2; 2) vitamin B6 in an amount greater than 7.5 mg; 3) magnesium citrate in an amount greater than 180 mg; and 4) potassium (such as in the form of potassium citrate) in an amount not exceeding 100 or 99 mg.

In addition to medicinal compositions, nutritional compositions comprise components as disclosed above. Thus, in one aspect, a medicinal composition is provided that includes an effective amount of vitamin K (i.e., K1 and/or K2) in combination with potassium citrate, magnesium citrate, and vitamin B6. In certain embodiments the composition contains an effective amount of MK4 and/or MK7. The composition also may optionally comprise vitamin K1. In a further aspect, nutritional compositions including in oral dosage forms such as tablets or capsules are provided that comprise 1) an effective amount of vitamin K2; 2) vitamin B6 in an amount greater than 7.5 mg; 3) magnesium citrate in an amount greater than 180 mg; and 4) potassium (such as in the form of potassium citrate) in an amount not exceeding 100 or 99 mg.

Certain compositions are preferably in an oral dosage form, such as a capsule or tablet. Other composition formulations also will be suitable such as a gel, powder, liquid, suspension or emulsion.

In one embodiment, a therapeutic composition is packaged in a multiple component container. Optionally, a first container component comprises an effective amount of vitamin K (i.e., K1 and/or K2) and a second container component comprises one or more of a citrate salt, magnesium oxide, vitamin B6, or a bicarbonate salt. Optionally, opening of the container admixes contents of the first and second container components. The container can be adapted to a variety of configurations. For instance, the container suitably may be a multiple component sachet. Alternatively, the container may be configured as a single dose vial.

In a further embodiment, the composition may be packaged in a multiple component container, where a first container component comprises an effective amount of vitamin K (i.e., K1 and/or K2) in one or more oral dosage forms and a second container component comprises one or more of a citrate salt, magnesium oxide, vitamin B6, or a bicarbonate salt in one or more oral dosage forms. Optionally, the one or more oral dosage forms of the first component and the one or more oral dosage forms of the second component are delivered together upon opening of the container.

Kits are also provided that suitably may comprise vitamin K (i.e., K1 and/or K2) and instructions for use of the vitamin K (i.e., K1 and/or K2) for treatment of hypercalciuria or nephrolithiasis or for promoting normal urine chemistry and composition. The instructions typically will be in written form, for example as presented on a package insert or a product label. The kit suitably also may comprise one or more of citrate salt, magnesium oxide, vitamin B6, or a bicarbonate salt. The kit may comprise a multiple component container as described above where a first container component comprises an effective amount of vitamin K2 and a second container component comprises one or more of a citrate salt, magnesium oxide, vitamin B6, or a bicarbonate salt. The kit also may comprise a multiple component container, where a first container component comprises an effective amount of vitamin K2 in one or more oral dosage forms and a second container component comprises one or more of a citrate salt, magnesium oxide, vitamin B6, or a bicarbonate salt in one or more oral dosage forms.

Other aspects of the invention are disclosed infra.

### DETAILED DESCRIPTION

In certain aspects, methods are provided to decrease urinary calcium in patients with hypercalciuria.

In one aspect, the present invention features methods of treating or preventing hypercalciuria or calcium nephrolithiasis using a compound of Formula (1) as that formula is specified below. In another aspect, treating a subject with calcium nephrolithiasis and any one or more of the following: hypercalciuria, hyperuricosuria, hyperoxaluria, hypocitraturia, or hypomagnesiuria in need may include administering vitamin K2 in combination with or associated with a citrate salt, magnesium oxide, vitamin B6, or a bicarbonate salt.

A compound used in the methods and compositions of the invention may be one or more selected from compounds that belong to vitamin K family. As discussed above, it has been found herein that one or more forms of vitamin K2 such as MK4 and MK7 can efficiently reduce urinary calcium excretion. Selective use of a particular vitamin K2 compound also may be advantageous. For example, one form of vitamin K2 may be more active in patients than another form. Accordingly, a combinational use of various vitamin K2 compounds (e.g. MK4 and MK7) may provide improved efficacy.

In one aspect, therapeutic compounds used in the present methods and compositions may be represented by the following formula (1).

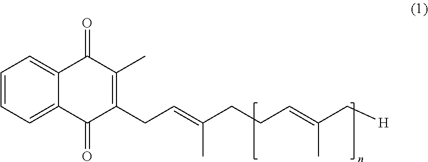

where n is an integer ranging from 0 to 20, or 1 to 10, or n is 3 (i.e. MK4) or 6 (i.e. MK7).

The compound of formula (I) contains unsaturated C5 chains, and thus, as generally known, the above compound comprising 2-methyl-1,4-naphthoquinone and unsaturated C5 chains is referred to as vitamin K2.

Meanwhile, among the vitamin K family, vitamin K1 contains 2-methyl-1,4-naphthoquinone with an aliphatic side chain, which can be excluded as a compound represented by formula (1).

As indicated, when n is 3, the compound is referred to MK4 as represented below.

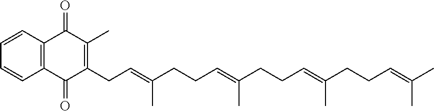 [SMILES: CC(C)=CCC/C(C)=C/CC/C(C)=C/CC/C(C)=C/CC1=C(C)C(=O)c2ccccc2C1=O]

When n is 6, the compound is referred to MK7 represented as follow:

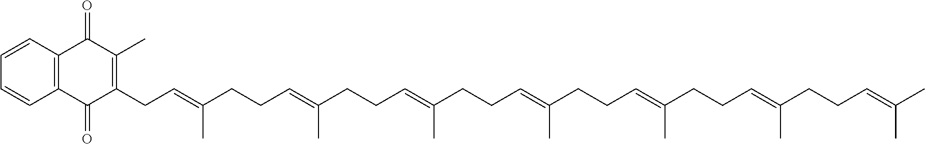 [SMILES: CC(C)=CCC/C(C)=C/CC/C(C)=C/CC/C(C)=C/CC/C(C)=C/CC/C(C)=C/CC/C(C)=C/CC1=C(C)C(=O)c2ccccc2C1=O]

In some embodiments, the compound of the invention may include a mixture of the compounds of formula (1).

Vitamin K2 as presented in formula (1) above, particularly MK4, can be produced by conversion of vitamin K1 in the testes, pancreas, and arterial walls or the like in a body. For example, aliphatic tails in vitamin K1 may be metabolically removed and unsaturated isoprenyl moieties may be attached to the quinone moiety to biosynthesize vitamin K2. However, without wishing to be bound to the theory, combinational use of vitamin K1 may increase internal dose of vitamin K2 via metabolic pathways in the body.

Accordingly, the compound of the invention may be mixed with vitamin K1 to enhance metabolic or therapeutic dose of the compound (vitamin K2) in a subject.

In some embodiments, as mentioned, a compound of the invention may be used as a mixture of compounds presented by formula (1). In certain embodiments, the compound may comprise at least MK4 or at least MK7. In certain embodiments, the compound may comprise a mixture of MK4 and MK7, without particular limitations to the mixing ratio. For example, the MK4 may be mixed with MK7 at a weight ratio of about 1 to 150, 1 to 100, 1 to 9, 1 to 4, 3 to 7, 2 to 3, 1 to 1, 3 to 2, 7 to 3, 4 to 1 or 9 to 1 or 100 to 1 or 150 to 1. In some embodiments, because MK7 has a longer half-life and greater bioavailability or biostability than MK4, a greater amount or ratio of MK4 may be included in the mixture, within the no observed adverse effect level (NOAEL).

The therapeutically effective dose of one or more compounds administered in accordance with the present invention may be determined as an amount of the administered compound sufficient to reduce calcium concentration in a urine sample of a subject by about 5% or greater, by about 10% or greater, by about 15% or greater, by about 20% or greater, by about 30% or greater, by about 40% or greater, by about 50% or greater, by about 60% or greater, by about 70% or greater, or by about 80% or greater, after administration for at least 24 hrs.

In certain embodiments, the therapeutically effective dose of MK4 may be about 100 mcg/kg/day, 200 mcg/kg/day, 300 mcg/kg/day, 400 mcg/kg/day, 500 mcg/kg/day, 600 mcg/kg/day, 700 mcg/kg/day, 800 mcg/kg/day, 900 mcg/kg/day, 1000 mcg/kg/day, 2 mg/kg body/day, 3 mg/kg body/day, 4 mg/kg body/day, 5 mg/kg body/day, 6 mg/kg body/day, 7 mg/kg body/day, 8 mg/kg body/day, 9 mg/kg body/day, 10 mg/kg body/day, 15 mg/kg body/day, 20 mg/kg body/day, 30 mg/kg body/day, 4 mg/kg body/day, 50 mg/kg body/day, 100 mg/kg body/day, 150 mg/kg body/day, 200 mg/kg body/day, 250 mg/kg body/day, 300 mg/kg body/day, 350 mg/kg body/day, 400 mg/kg body/day, 450 mg/kg body/day, or 500 mg/kg body/day. In some embodiments, the therapeutically effective dose of MK4 may be determined at a maximum dose where no observed adverse effect level (NOAEL) is found.

In certain embodiments, the therapeutically effective dose of MK7 may be about 1 mcg/kg/day, 2 mcg/kg/day, 3 mcg/kg/day, 4 mcg/kg/day, 5 mcg/kg/day, 6 mcg/kg/day, 7 mcg/kg/day, 8 mcg/kg/day, 9 mcg/kg/day, 10 mcg/kg/day, 15 mcg/kg/day, 20 mcg/kg/day, 30 mcg/kg/day, 40 mcg/kg/day, 50 mcg/kg/day, 60 mcg/kg/day, 70 mcg/kg/day, 80 mcg/kg/day, 90 mcg/kg/day, 100 mcg/kg/day. In some embodiments, the therapeutically effective dose of MK7 may be determined at a maximum dose where no observed adverse effect level (NOAEL) is found.

In some embodiments, one or more vitamin K2 compounds may be mixed with vitamin K1 to enhance metabolic or therapeutic dose of the compound in the body. The mixing ratio of one or more vitamin K2 compounds and vitamin K1 suitably can vary widely. In certain embodiments, a therapeutically effective dose of vitamin K1 in combination with MK4 and/or MK7 may be about 1 mcg/kg/day, 2 mcg/kg/day, 3 mcg/kg/day, 4 mcg/kg/day, 5 mcg/kg/day, 6 mcg/kg/day, 7 mcg/kg/day, 8 mcg/kg/day, 9 mcg/kg/day, 10 mcg/kg/day, 15 mcg/kg/day, 20 mcg/kg/day, 30 mcg/kg/day, 40 mcg/kg/day, 50 mcg/kg/day, 60 mcg/kg/day, 70 mcg/kg/day, 80 mcg/kg/day, 90 mcg/kg/day, 100 mcg/kg/day. In some embodiments, the therapeutically effective dose of vitamin K1 may be determined at a maximum dose where no observed adverse effect level (NOAEL) is found.

In some embodiments, for the use of treating calcium nephrolithiasis and any one or more of the following: hypercalciuria, hyperuricosuria, hyperoxaluria, hypocitraturia, or hypomagnesiuria, the compound may be administered together with one or more selected from the group consisting of a citrate salt such as potassium citrate, magnesium citrate or calcium citrate; magnesium oxide; vitamin B6; or a bicarbonate salt, and combinations thereof.

The subject to be administered with one or more compounds as disclosed herein is suitably a mammal, or particularly a human. In certain embodiments, the subject or the human to be treated may suffer from hypercalciuria. For instance, a human subject may excrete, per 24 hour period, greater than about 50 mg of calcium, greater than about 100 mg of calcium, greater than about 150 mg of calcium, or greater than about 200 mg of calcium.

Accordingly, in some embodiments, the method of treating hypercalciuria may further comprise a step of selecting the subject suffering from hypercalciuria, particularly the subject excreting per 24 hour period, greater than about 50 mg of calcium, greater than about 100 mg of calcium, greater than about 150 mg of calcium, greater than about 200 mg of calcium, or greater than 250 mg of calcium. In certain embodiments, the subject may excrete about 200 mg of calcium per 24 hour period.

In some embodiments, the therapeutically effective dose of the compound may be administered in combination with known anti-nephrolithiasis treatments, to reduce the risk of nephrolithiasis. Exemplary anti-nephrolithiasis treatments include, e.g., administration of non-steroidal anti-inflammatory drugs (NSAIDs), optionally in the form of diclofenac IM or PR, for the relief of the severe pain of colic; administration of parenteral morphine (optionally excluding pethidine); administration of antiemetics and rehydration therapy, if needed; extracorporeal shock wave lithotripsy (ESWL); administration of medical expulsive therapy, e.g., calcium-channel blockers (e.g., nifedipine) or alpha-blockers (e.g., tamsulosin), optionally including administration of a corticosteroid such as prednisolone, optionally when an alpha-blocker is used, to facilitate the passage of the stone; and surgical removal.

The therapeutically effective dose of the compound can be administered to the subject by a variety of administration routes. Oral or topical administration will be typically preferred although other administration protocols also may be utilized as parenteral, sublingual, or via an implanted reservoir. In some embodiments, the compound may be formulated for administering purposes in a capsule, a tablet, a gel, a powder, liquid, suspension or emulsion.

As discussed, therapeutic compositions are also provided that include one or more compounds as disclosed herein optionally with a pharmaceutically acceptable carrier.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

In one preferred aspect, the compound may be formulated for administering purposes in a capsule, a tablet, a gel, a powder, liquid, suspension or emulsion; however, the administering methods may not be particularly limited.

In some embodiments, the therapeutically effective dose of the compound may be administered orally, parenterally, buccal, sublingually, or via an implanted reservoir

The compound(s) can be included in a kit, container, pack, or dispenser together with instructions for administration. For instance, the kit may contain a product label or written package insert that discloses use of the composition for treating including prophylaxis of hypercalciuria and/or nephrolithiasis. For a nutritional composition or supplement that comprises a vitamin K as disclosed herein, the product label or insert may suitably disclose use of the composition to promote normal urine chemistry and composition.

In one preferred aspect, the formulation of the invention comprising the compounds of formula (1) may be used in combination with or include one or more other therapeutic agents or dietary or nutritional supplements and may be administered either sequentially or simultaneously by any convenient route in separate or combined pharmaceutical or nutritional compositions. As used herein, combination of two or more compounds may refer to a composition wherein the individual compounds are physically mixed or wherein the individual compounds are physically separated. A combination use encompasses administering the components separately to produce the desired additive, complementary or synergistic effects. In certain exemplary embodiments, the compound and the agents (e.g. potassium citrate, magnesium citrate, magnesium oxide, vitamin B6, calcium citrate, a bicarbonate salt and combinations thereof) are physically mixed in the composition. In additional exemplary embodiments, the compound and the agent are physically separated in the composition.

In an exemplary embodiment, an additional bioactive agent may be added to a formulation comprising the compound of the invention. Alternatively, the formulation of the invention may further comprise other drug components for complicated disease treatment or prevention with combined use.

### EXAMPLES

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1: Treatment of Hypercalciuria Using Vitamin K2 Alone or in Combination with Other Treatments

Patients with hypercalciuria who were not on anticoagulants were routinely recommended vitamin K2 supplementation. Eight patients with nephrolithiasis that were found to have hypercalciuria (>200 mg/24 hr) on baseline 24 hour urine testing with or without any other stone risk factor took one of the commercially available forms of vitamin K2, MK7 supplements. All patients also received standard treatments for any other stone risk factor found in their work-up. As is routine, these patients were asked to repeat the 24 hr urine test, to evaluate response to treatment. Of the eight patients, seven exhibited an average 49% decrease in the excretion of calcium in the urine. This compared favorably to the 30-50% expected decrease in calcium when a thiazide diuretic is prescribed. See Martins, M. C., et al., Br J Urol, 1996. 78(2): p. 176-80. Furthermore, the calcium lowering effect of vitamin K2 was independent of the presence and treatment of other stone risk factors. As a comparison, in a study where vitamin K1 was evaluated for its ability to reduce urinary calcium in hypercalciuric subjects, only 60% of the subjects had a decrease in urinary calcium, and the average reduction in urinary calcium in patients exhibiting a similar level of pre-treatment hypercalciuria to that of the vitamin K2-treated population presented here was less than 15%.

Table 1 shows the effect on 24 hour urine calcium measurements of vitamin K2 in the form of MK7 alone or in addition other nephrolithiasis treatments in patients on no prior nephrolithiasis preventive therapy and in patients on stable background nephrolithiasis prevention therapy.

TABLE 1

| | | | Before Vitamin K | | On Vitamin K | | |
|---|---|---|---|---|---|---|---|
| | | | | Urine Calcium | | Urine | |
| | | M/ | | amount | | Calcium | % |
| Case # | Age | F | Regimen | (mg/24 hr) | Regimen | (mg/24 hr) | diff |
| 1 | 51 | F | | 650 | allopurinol 300 mg daily | 262 | −60 |
| | | | | | MK7 100 mcg daily | | |
| 2 | 59 | M | | 312 | KCitrate 99 mg bid | 145 | −54 |
| | | | | | MgOxide 90 mg bid | | |
| | | | | | MgCitrate 90 mg bid | | |
| | | | | | Vitamin B6 7.5 mg bid | | |
| | | | | | allopurinol 300 mg daily | | |
| | | | | | MK7 50 mcg bid | | |
| 3 | 61 | F | | 321 | KCitrate 200 mg daily | 158 | −51 |
| | | | | | MK7 120 mcg bid | | |
| 4 | 59 | M | KCitrate 1080 mg bid | 320 | KCitrate 1080 mg tid | 124 | −61 |
| | | | | | MK7 100 mcg bid | | |
| 5 | 42 | F | allopurinol 300 mg | 326 | allopurinol 100 mg daily | 225 | − 31 |
| | | | daily | | | | |
| | | | KCitrate 99 mg bid | | KCitrate 99 mg bid | | |
| | | | MgOxide 90 mg bid | | MgOxide 90 mg bid | | |
| | | | MgCitrate 90 mg bid | | MgCitrate 90 mg bid | | |
| | | | Vitamin B6 7.5 mg bid | | Vitamin B6 7.5 mg bid | | |
| | | | | | MK7 120 mcg bid | | |
| 6 | 67 | M | HCTZ 12.5 mg daily | 332 | HCTZ 12.5 mg daily | 340 | +2 |
| | | | | | KCitrate 99 mg bid | | |
| | | | | | MgOxide 90 mg bid | | |
| | | | | | MgCitrate 90 mg bid | | |
| | | | | | Vitamin B6 7.5 mg bid | | |
| | | | | | MK7 120 mcg bid | | |
| 7 | 58 | F | KCitrate 99 mg bid | 314 | KCitrate 99 mg bid | 84 | −73 |
| | | | MgOxide 90 mg bid | | MgOxide 90 mg bid | | |
| | | | MgCitrate 90 mg bid | | MgCitrate 90 mg bid | | |
| | | | Vitamin B6 7.5 mg | | Vitamin B6 7.5 mg bid | | |
| | | | bid | | HCTZ 25 mg bid | | |
| | | | HCTZ 25 mg bid | | prednisone 5 mg daily | | |
| | | | prednisone 5 mg daily | | vitD3 1000 mg daily | | |
| | | | vitD3 1000 mg daily | | allopurinol 300 mg daily | | |
| | | | allopurinol 300 mg | | MK7 150 mcg daily | | |
| | | | daily | | | | |
| 8 | 74 | F | CaCitrate 630 mg bid | 376 | CaCitrate 630 mg tid | 321 | −15 |
| | | | | | KCitrate 99 mg bid | | |
| | | | | | MgOxide 90 mg bid | | |
| | | | | | MgCitrate 90 mg bid | | |
| | | | | | Vitamin B6 7.5 mg bid | | |
| | | | | | MK7 120 mcg daily | | |
| mean | | | | | | | −43 |
| median | | | | | | | −52 |
| standard deviation | | | | | | | 26 |

As shown in Table 1, ingestion of MK7 (100-150 mcg daily or bid) for at least 3 weeks resulted in a marked decrease in 24 hour urine calcium. 5 out of 8 patients exhibited a decrease in urinary calcium of greater than about 50%. The average decrease in urine calcium in all eight patients was about 43%. No patient exhibited a significant increase in urinary calcium and only one showed no response (2% increase). (Table 1)

In particular, when the vitamin K was administered together with other agents, for example, in cases 1, 2 and 3 urinary calcium excretion decreased >50%. Because low levels of potassium citrate supplementation, as in cases 2 and 3, are not expected to reduce urinary calcium, the marked decrease in urinary calcium was likely due to the action of vitamin K2. Because allopurinol, as in case 1, and calcium citrate have been shown to increase urinary calcium, their use does not explain the decrease in urinary calcium observed with the addition of vitamin K2. The rest of the cases had the vitamin K2 added to their stable background regimen.

### Example 2: Combined Use of MK4 and MK7

Table 2 shows the effect on 24 hour urine calcium of a change in vitamin K2 therapy from low dose of MK7 daily to high dose of MK4 daily. One patient changed the dose of vitamin K2 from taking MK7 at a dose of 120 mcg daily to taking MK4 at a dose of 15 mg daily, and a significant additional decrease of about 40% in calcium excretion was noted in her 24 hour urine calcium.

TABLE 2

| | | | | High Dose MK4 | | |
|---|---|---|---|---|---|---|
| | | | | | Urine | |
| | M/ | Low Dose MK7 | | | Calcium | % |
| Age | F | Regimen | UCa (mg/24 hr) | Regimen | (mg/24 hr) | difference |
| 74 | F | CaCitrate 630 mg tid | 321 | CaCitrate 630 mg tid | 191 | −40 |
| | | KCitrate 99 mg bid | | KCitrate 99 mg bid | | |
| | | MgOxide 90 mg bid | | MgOxide 90 mg bid | | |
| | | MgCitrate 90 mg bid | | MgCitrate 90 mg bid | | |
| | | Vitamin B6 7.5 mg bid | | Vitamin B6 7.5 mg bid | | |
| | | MK7 120 mcg daily | | MK4 15 mg daily | | |

### Comparative Example

Table 3 shows magnitude of the normal variation between two 24 hour urine calcium measurements in a group of control patients with no change in nephrolithiasis prevention therapy, for example, without supplementation with vitamin K2. Particularly, on average, the 24 hour urine calcium was reduced about 4% with a median decrease of 8% and a standard deviation between patients of 24%. The largest difference between two collections was 31%.

TABLE 3

| | | M/ | First | Second | % |
|---|---|---|---|---|---|
| Case # | Age | F | Collection | Collection | difference |
| 1 | 55 | M | 492 | 367 | −25 |
| 2 | 58 | F | 327 | 226 | −31 |
| 3 | 45 | M | 353 | 325 | −8 |
| 4 | 68 | M | 371 | 275 | −26 |
| 5 | 60 | M | 289 | 323 | +12 |
| 6 | 48 | F | 289 | 355 | +23 |
| 7 | 33 | M | 350 | 438 | +25 |
| average | | | | | −4 |
| median | | | | | −8 |
| standard deviation | | | | | 24 |

Thus, results observed above for treatment(s) with vitamin K were well outside the range of typical day-to-day variation of urine calcium levels in a subject.

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All documents mentioned herein are herein incorporated by reference herein.

1. A method of treating hypercalciuria in a mammal comprising administering an effective amount of vitamin K2 to the mammal.

2. The method of claim 1 wherein an effective amount of MK4 is administered to the mammal.

3. The method of claim 1 wherein an effective amount of MK7 is administered to the mammal.

4. The method of claim 1 wherein vitamin K1 is co-administered to the mammal.

5. The method of claim 1 wherein the vitamin K is administered to the mammal in conjunction with one or more of the following: a citrate salt, magnesium oxide, vitamin B6, and a bicarbonate salt.

6. A method of treating hypercalciuria in a mammal comprising administering an effective amount of vitamin K1 in conjunction with one or more of the following: a citrate salt, magnesium oxide, vitamin B6, and a bicarbonate salt.

7. The method of claim 1 wherein the mammal is identified as suffering from hypercalciuria.

8. The method of claim 1 further comprising selecting a human that excretes greater than 200 mg of calcium in the urine per 24 hour period.

9-13. (canceled)

14. A method of treating calcium nephrolithiasis in a mammal in need thereof comprising administering to said mammal an effective amount of vitamin K.

15-26. (canceled)

27. The method of claim 1 wherein the subject is administered vitamin K in an oral dosage form that comprises greater than 99 mg of potassium.

28-29. (canceled)

30. A therapeutic composition comprising an effective amount of vitamin K, potassium citrate, magnesium citrate, and vitamin B6.

31-46. (canceled)

47. A nutritional composition comprising an effective amount of vitamin K, potassium citrate, magnesium citrate, and vitamin B6.

48. The nutritional composition of claim 47 wherein the composition is at least substantially free of calcium.

49. The nutritional composition of claim 47 or 48 wherein the composition contains an effective amount of MK4.

50. The nutritional composition of claim 47 wherein the composition contains an effective amount of MK7.

51-63. (canceled)

64. A kit comprising vitamin K2 and instructions for use of the vitamin K2 for treatment of hypercalciuria, calcium nephrolithiasis, or for promoting normal urine chemistry or composition, or
A kit comprising vitamin K1 and instructions for use of the vitamin K1 for treatment of calcium nephrolithiasis or for promoting normal urine chemistry or composition.

65. (canceled)

66. The kit of claim 64 wherein the kit further comprises one or more of the following: a citrate salt, magnesium oxide, vitamin B6, and a bicarbonate salt.

67-72. (canceled)
